(19) United States
(12) Patent Application Publication
(10) Pub. No.: US 2020/0015885 A1
(43) Pub. Date: Jan. 16, 2020

(54) CATHETER LEAD AND METHOD OF MANUFACTURE THEREOF

(71) Applicant: CATHRX LTD, New South Wales (AU)

(72) Inventor: Roman Greifeneder, New South Wales (AU)

(21) Appl. No.: 16/489,126

(22) PCT Filed: Mar. 15, 2018
(86) PCT No.: PCT/AU2018/050241

§ 371 Date: Aug. 27, 2019

(30) Foreign Application Priority Data
Mar. 22, 2017 (AU) 2017901015

(51) Int. Cl. A61B 18/14; A61M 25/01; A61B 5/00; A61M 25/00; A61M 25/06; B29C 63/38

(52) U.S. Cl. CPC A61B 18/1492; A61M 25/0136; A61B 5/6852; A61M 25/0013; A61B 2018/00077; A61M 25/0662; B29C 63/38; A61B 2018/1407; A61M 25/001

(57) ABSTRACT

Catheter lead for a procedure comprising a tubular member with at least one conductive wire. The tubular member having a proximal end and a distal end with the distal end of the catheter comprising at least one electrode in communication with the conductive wire and wherein the at least one electrode comprises a conductive polymeric material.

### TECHNICAL FIELD

The present invention relates to a catheter lead and a method of manufacture thereof. More particularly, the present disclosure is directed towards a catheter lead which is conductive and/or radiopaque and preferably comprises conductive plastics.

### BACKGROUND

A number of catheter leads are known which allow for cardiac surgery to take place. These leads may allow for ablation, diagnostics or other procedures to be conducted. However, there are a number of problems with existing leads.

Known catheter leads comprising electrodes are generally formed with biocompatible metal components which are adapted to be inserted into a patient. These leads generally have electrodes with are elevated above or raised relative to the catheter lead surface. Other known leads are generally formed with pull-wires which are generally undesirable as they are fixed to the distal end of the catheter lead or only form limited shapes when activated. Further, catheters equipped with pull-wires generally are not capable of having a stylet to replace the pull-wire, nor is the function of a pull-wire an equivalent to that of a stylet. More particularly, the pull-wire is similar to a tendon, in which applying tension to a pull-wire causes the catheter lead attached thereto to deform. However, these types of deflection means can only impart a shape at the location in which the pull-wire is attached and activated, which can cause steering problems in use.

Most catheters known are also incapable of being used more than once, and this is generally a large expense for procedures. Further accidental contact with a non-sterile surface may cause an entire catheter to be discarded without use which can cause substantial expense to a hospital or clinician. In addition, discarding catheters may also result in expensive rare earth metals to be discarded along with a catheter lead, or the electronics of the catheter handle to be discarded despite no contact with biological matter. Further, replacing components of a catheter lead and/or catheter handle is generally unknown in the art.

Further, it is difficult with known catheters to form a desired shape at a desired portion of the catheter. This can cause the lead to snag anatomy in use, which can cause severe lesions, internal bleeding and undesired medical complications.

U.S. Pat. No. 5,611,777 A (Bowden et al.) discloses a steerable catheter which includes a control handle having a tubular housing with a rotatable thumbwheel and a slideblock for effecting deflection of the catheter. The radius of curvature of the tip portion of the catheter, when deflected, depends upon how far distally into the deflectable tip portion the radius adjusting wire has been advanced by the user. This document does not disclose or suggest a stylet, and only suggests an ring electrode formed from platinum. Further, this document provides complex internal components, such as a pull sire mechanism which typically has a number of draw backs. In addition, the catheter of Bowden cannot be reused or reprocessed.

U.S. Pat. No. 5,524,337 A (Houser et al.) there is disclosed a method of securing ring electrodes onto catheter. This document relates to catheters having rigid ring electrodes secured thereto and a method for securing such electrodes to a catheter body via clamping. More particularly, the invention relates to such electrodes and methods related to catheters intended for endocardial mapping and ablation systems.

Referring to U.S. Pat. No. 5,029,585 (Lieber et al.) discloses intralumen electrodes for use with medical catheters. The electrodes are made of a conductive polymeric material that is introduced into the lumen of a catheter through an opening cut in the peripheral wall in the catheter. A conductive lead threaded through the lumen of the catheter terminates in a distal end at the opening in the catheter and is completely embedded within the polymeric material introduced into the opening, thereby establishing electrical contact between the conductive polymeric electrode and the conductive lead. The conductive polymeric material fills the opening adhering to the walls of the catheter tube, thereby ensuring secure, long lasting attachment. Again, this document discloses rigid ring electrodes around a catheter lead.

Turning to U.S. Pat. No. 6,652,506 (Bowe et al.) there is disclosed a catheter handle includes a steering controller with a self-locking mechanism to be used in conjunction with a steerable catheter shaft. A compression spring portion of the self-locking mechanism is located between the steering controller and a handle shell and causes alternating protrusions and recesses on the steering controller and on the handle shell to engage, thus locking the steering controller into a fixed position with the handle shell. Through a single-handed operation, an operator enables steering controller rotation by applying a force to the steering controller, which disengages the steering controller from the handle shell. The operator then adjusts the profile of a distal-end region of the catheter by rotating the steering controller. When the desired profile of the distal-end region of the catheter has been obtained, the operator removes the force from the steering controller and the spring decompresses to reengage the steering controller with the handle shell. The device as described in this comprises a number of problems, such a pull-wire and also a catheter handle which comprises a one way press fit. Further, the internal components of the device comprise undesirable and complex rotational means to manipulate a pull wire which leads to very limited deflection shapes, such as a ‘J-shape’ of the distal-end region of the catheter.

Referring to U.S. Pat. No. 5,545,200 A (West et al.) there is disclosed an electrophysiology catheter with a manipulator wire is coupled to the distal end of the deflectable tip, whereby the deflectable tip may be deflected by axial force applied to the manipulator wire. Further, the distal end of the deflectable tip must remain in a substantially constant axial position, preferably in a plane perpendicular to the longitudinal axis. This document therefor comprises similar problems to the above documents. In addition, the electrodes of the outer surface of the catheter are generally disposed at an elevated level, relative to that of the lead.

Another known document is U.S. Pat. No. 6,263,224 B1 (West). This document discloses a multifunction and radial deflection wires which extend from a catheter shaft and into a handle. Again, this disclosure comprises pull-wires which act like tendons to only effect movement of the distal end of the catheter. Further, while there are a number of exploded views in this document, there is no disclosure or suggestion of a reusable catheter let alone a catheter which can be separated to access the components internal to the device. The internal components of the device are also complex and do not allow for mounting of particular components as essential features to operate the device fill the internal cavity of the handle.

There may be a need to develop a catheter which provides an advantage over the prior art which may allow for reduced manufacturing costs in relation to known catheters. There may also be a need to develop a catheter which can be more readily manipulated in use, as desired by a clinician.

Any discussion of the prior art throughout the specification should in no way be considered as an admission that such prior art is widely known or forms part of common general knowledge in the field.

### SUMMARY

### Problems to be Solved

It may be advantageous to provide for a catheter and/or catheter lead which is low cost to manufacture.

It may be advantageous to provide for a catheter lead which comprises electrodes which are formed from polymer, or a polymer and metal.

It may be advantageous to provide for a catheter lead which comprises electrodes which may flex or bend.

It may be advantageous to provide for a catheter lead which comprises electrodes which may be elastically deformed during use.

It may be advantageous to provide for a catheter which can be substantially free of metallic materials which is exposed to the anatomy of a patient.

It may be advantageous to provide for a catheter lead which is formed with polymer electrodes to reduce manufacturing cost.

It may be advantageous to provide for a catheter lead which can be at least one of recycled, reused or reprocessed.

It is an object of the present invention to overcome or ameliorate at least one of the disadvantages of the prior art, or to provide a useful alternative.

### Means for Solving the Problem

A first aspect of the present disclosure may relate to a catheter lead for a procedure. The catheter lead comprising a tubular member with at least one conductive wire. The tubular member having a proximal end and a distal end. The distal end of the catheter comprising at least one electrode in communication with the conductive wire and wherein the at least one electrode comprises a conductive polymeric material.

Preferably, the at least one conductive wire is helically wound. Preferably, the at least one conductive wire is helically wound around the tubular member. Preferably, the tubular member is formed from the helically wound at least one conductive wire. Preferably, the electrode is adapted to deform. Preferably, the electrode is adapted to elastically deform. Preferably, the electrode is formed from conductive polymeric material. Preferably, the distal end of the catheter comprises at least two electrodes in which an intermediate layer disposed between the at least two electrodes. Preferably, the intermediate layer abuts a side of the at least two electrodes. Preferably, the outer surface of the intermediate layer and the outer surface of the at least two electrodes is flush. Preferably, the outer surface of the catheter lead is molded by a heat-shrink layer.

A further aspect of the present disclosure may relate to a method of manufacturing a catheter sheath. The method comprising the steps of cutting an aperture in a tubular member to expose a conductive wire; applying an electrode over the conductive wire; applying a heat-shrink layer with an intermediate layer over the tubular member and the electrode; applying heat to the heat-shrink layer such that the intermediate layer transitions from a solid state to a fluid state, the heat further causing the heat-shrink layer to radially shrink around the intermediate layer; and removing the heat-shrink layer after the intermediate layer has solidified.

Preferably, the aperture the expose the conductive wire is filled with a conductive material. Preferably, at least one tacking aperture is formed in the tubular member. Preferably, a conductive band is disposed between the electrode and the conductive wire. Preferably, the outer surface of the intermediate layer is moulded to be substantially level with the outer surface the electrode. Preferably, the heat-shrink layer is used as a mould to mould the intermediate layer.

In the context of the present invention, the words “comprise”, “comprising” and the like are to be construed in their inclusive, as opposed to their exclusive, sense, that is in the sense of “including, but not limited to”.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1 illustrates a side view of an embodiment of a catheter with a catheter sheath extending from a distal end of a catheter handle.

FIG. 2 illustrates an embodiment of a portion of a tubular member of a catheter with conductive wires helically wound around said tubular member.

FIG. 3 illustrates an embodiment of a portion of a process for manufacturing a catheter sheath with a tubular member formed from helically wound wires.

FIG. 4 illustrates a portion of a process of the embodiment of FIG. 3, in which taking slots are formed through the tubular member.

FIG. 5 illustrates a portion of a process of the embodiment of FIG. 3, in which a conductive slot is formed in the tubular member.

FIG. 6 illustrates a portion of a process of the embodiment of FIG. 3, in which the conductive slot is filled with a conductive material.

FIG. 7 illustrates a portion of a process of the embodiment of FIG. 3, in which a conductive band is disposed over the conductive slot.

FIG. 8 illustrates a portion of a process of the embodiment of FIG. 3, in which a polymer electrode is disposed relatively over the conductive slot.

FIG. 9 illustrates a portion of a process of the embodiment of FIG. 3, in which a the outer surface of the catheter sheath is formed.

FIG. 10 illustrates an embodiment of an example of a catheter handle which is adapted to be separable and reattachable, and also receive a catheter sheath of the present invention.

FIG. 11 illustrates an embodiment of the catheter sheath in which the distal end of the sheath is adapted to project beyond the distal end of the stylet.

### DESCRIPTION OF THE INVENTION

Preferred embodiments of the invention will now be described with reference to the accompanying drawings and non-limiting examples.

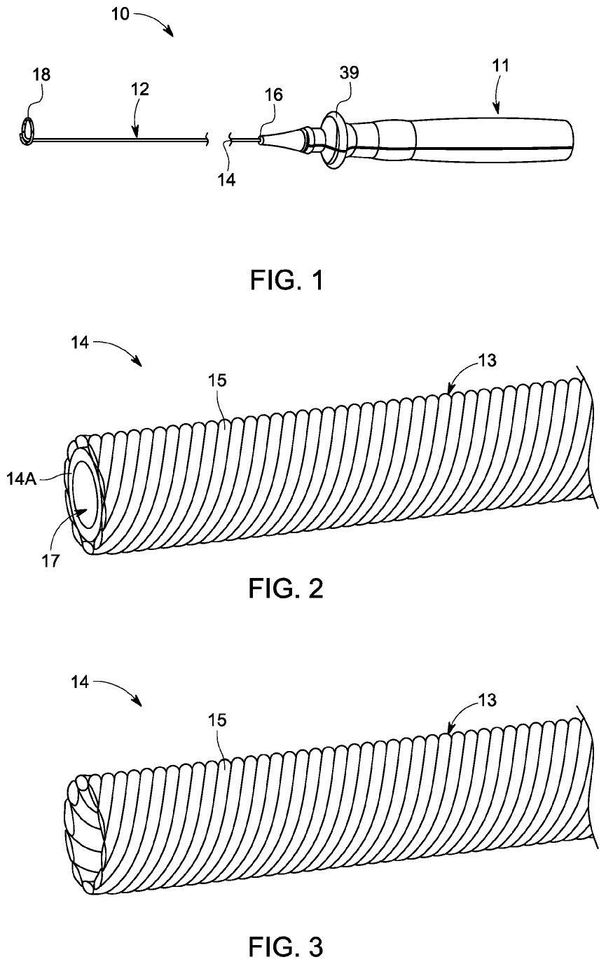

Referring to the embodiment of FIG. 1, the present disclosure is directed broadly towards a catheter 10 and a method of manufacture of components therefor. More particularly the present disclosure may concern a novel method of manufacture of a catheter lead 12. The catheter 10 preferably includes a handle 11 and an elongate catheter lead 12 extending from a distal end of the handle 11. The catheter lead 12 can be formed with a generally tubular member 14 having a proximal end 16 connectable with the handle 11 and a distal end 18 having one or more electrodes 20 attached to the tubular member 14. It will be appreciated that the tubular member 14 may instead be replaced with a member without a lumen (not shown).

While there are numerous references to a catheter sheath 12 throughout the specification, the term ‘catheter sheath 12’ may be used interchangeably with ‘catheter lead 12’. It will be appreciated that the term ‘lead’ need not be a ‘sheath’ such that it does not include a lumen 17 or other aperture to receive a stylet or fluid, for example.

An embodiment of a tubular member 14 used for manufacturing a portion of the catheter sheath 12 is shown in FIG. 2. The tubular member 14 is shown as being formed with a tubular element 14A with a plurality of wires 15 would helically (helical winding 13) around the tubular element 14A. The wires may be embedded, pressed into, glued, fixed, adhered or otherwise attached to the tubular element 14A. Embedding the wires 15 is a preferred method as this will substantially or completely fill the voids between the wires the tubular element 14A.

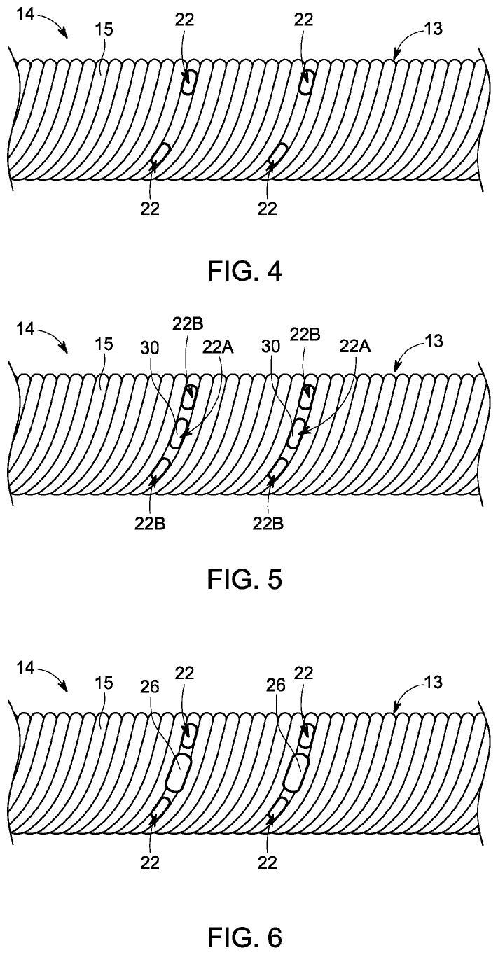

Referring to FIG. 3, there is illustrated a generally tubular member 14 which may be formed from at least one three-dimensional spiralled wire 15. The spiralled wire 15 is preferably of a constant diameter such that it forms a helix, but may also be formed with a tapered portion or portions with varying diameters if desired. Tapered or spiralled diameters may be advantageously used at the distal end of a catheter sheath to form a tip portion of the catheter sheath 12. However, the main portion of the tubular member 14 will preferably be of a constant diameter. Each of the wires 15 forming a helix will have a desired pitch spacing which preferably corresponds to an adjacent wire pitch. If a single wire is used, the wire comprises a plurality of helix pitches (complete turns of a helix) in which each helix pitch abuts at least one corresponding helix pitch relatively above and/or below, however this is with the exception of the formation of a spiral or tapered portion. If multiple wires 15 are used to form the tubular member 14, the helix angle to form the pitch of each wire may correspond to the number of wires 15 being used, such that each wire 15 forming the tubular member 14 may form an abutting relationship with an adjacent wire. In an example of a quadruple helix, if wires ABCD (not labelled in the Figures) are used, wire A will abut wires B and D, wire B will abut wires A and C, wire C will abut wires B and D, and wire D will abut wires C and A. It will be appreciated that the wires need not abut for the entire length of the tubular member 14. The helix angle may be in the range of 0° to 90°, but more preferably in the range of 25° to 50°, but even more preferably in the range of 30° to 45°. The helix angle may also impart a desired torsional property, flexure property or bending property to the catheter lead 12. Any number of wires 15 may be used to form the tubular sheath 14. At least one of the wires 15 in the helix 13 can be used as a conductor 30 to electrically connect at least one electrode to a power source, such as an RF source.

The electrodes 20 are preferably disposed at the distal end 18 of the catheter sheath 12 which is the end designated to be inserted into a patient. The electrodes 20 of the catheter sheath can be disposed in a predetermined array which may be an array with predetermined axially spaced intervals between the proximal end 16 and the distal end 18 of the tubular member 14. The array of electrodes 20 may only need be on a portion of the catheter sheath 12 which is preferably at and/or near to the distal end portion of the catheter sheath 12. The axially spaced intervals may be uniformly spaced or may have an irregular spacing based on a desired application. The desired application may be for sensing, diagnostics, mapping and/or may ablation. Ablation of tissue may refer to ablation of tissue of an organ, such as a heart, lung, liver or kidney.

Electrodes 20 of different shapes may be used; however the most preferred shape is an annular ring electrode 20 or portion of an annulus. The inner diameter of each ring electrode 20 may approximate the outer diameter of the tubular member 14 so that each ring electrode 20 is a snug fit about an external surface of the tubular member 14. However, the tubular member 14 have also have a depression, incision, cut, or deformation in which the ring electrode may be positioned within to be fixed to said tubular member 14. If this is the case, the ring electrode will have an inner diameter substantially corresponding to the depression, incision, cut or deformation.

Preferably, the ring electrodes 20 are formed from a conductive polymer. The polymer may have the advantage of being at least one of radiopaque, conductive and/or flexible. The polymer material may also have the advantage of being melted, fused, or bonded onto the tubular member 14 of the catheter sheath, or may be deposited in a depression or cut formed in the tubular member 14. Melting the electrode 20 may allow for a uniform flow of the electrode into place. Heating to cause a flow of the electrode 20 may allow for the formation of a substantially linear outer surface, in which the electrode 20 outer diameter is substantially the same as that of the catheter sheath outer diameter when manufacture is complete. The uniform diameter or linear surface of the catheter assists with prevention of biological materials being deposited as depressions or deformations in the catheter lead are essentially eliminated, and thus improving safety. Further, if a heat-shrink material is used the heat-shrink material and the electrode may be heated simultaneously to be fixed in a desired location on the tubular member 14 to form the catheter sheath 12.

Although, in a further embodiment the ring electrode 20 is a ring comprising platinum, for example a platinum-iridium ring, but may be made of other suitable biocompatible conductive materials. A mixture of metal and polymer may be used to reduce the overall production costs and allow for easier recycling after the catheter has been used, if recycling is desired or allowed by regulation. However, it will be appreciated that constructing the electrode 20 from an electron-conductive polymer may have a number of desired advantages. Optionally, a metal may be used for the electrode which is covered by a conductive polymer coating, in which the metal could include a typically non-biocompatible metal as the metal will not come into contact with the patient due to being enclosed by the polymer coating and the tubular member 14. It is preferred that if a polymer coating is used the metal is a biocompatible material, although this is optional.

A preferred method of manufacturing the catheter sheath 12 will now be described. In one embodiment, the catheter sheath 12 may be manufactured with a tubular member 14 formed with at least one conductive wire 15 wound around the tubular member 14. The tubular member 14 will form a lumen of the catheter sheath 12 which may be adapted to receive a manipulation means, such as a stylet. The tubular member 14 may instead have a plurality of wires 15 helically wound 13 around the tubular member 14, with at least one of the plurality of wires 15 being a conductive wire 30. Preferably, the tubular member 14 is formed from at least one helically wound wire 15, but preferably comprises a plurality of helically wound wires 15. The conductive wire 30 may instead be embedded in the tubular sheath 14, or may be disposed within the lumen 17 of the tubular member 14. While the tubular member 14 may be separately formed with conductive wires 15 disposed on said tubular member 14, reference will herein be made with respect to tubular member 14 being formed from a helically winding 13 of wires 15. The ‘helically winding of wires’ may be referred to herein as ‘wound wires’, although wound wires may not need be helically wound if desired. It will be appreciated that in at least one embodiment the wound wires 15 can be a series of conductive wires and non-conductive wires in any predetermined configuration or sequence. In a non-limiting example, the predetermined configuration may be one conductive wire with two non-conductive wires either side of the conductive wire. Each of the conductive wires preferably comprises a layer of insulation or outer jacket.

Referring to FIGS. 4 and 5, at least one aperture 22 is formed in the tubular member 14 and may be used to expose a conductive element of a wire 15. To expose a conductive element of a wire, the wire 15 will either be embedded in the tubular member 14 or will have a layer of insulation or outer jacket. The at least one aperture 22 may be formed by cutting, melting, burning, boring, or any other conventional means for forming an aperture 22. Preferably a laser is used to form the at least one aperture 22.

If the tubular member 14 is formed with helically wound wires 15 in which at least one wire is a conductive wire 30, the outer jacket or insulation of the wound wires 15 may have the aperture 22A formed therein to expose a selected conductive wire 30 within the tubular member 14. A plurality of apertures 22 may be formed along the length of the tubular member 14 which corresponds to the locations in which electrodes 20 are to be located. Apertures 22 may also be used as tacking locations 22B, which allow electrodes 20 to be tacked onto the tubular sheath 14. Each of the apertures 22 can be formed in the same wound wire 15 at desired intervals along the length of the wire 15. Apertures 22 may instead be formed a plurality of wires 15 in the tubular member 14. If apertures 22 are formed in different conductors 30 of the wound wires 15, each conductive wire 15 may be adapted to be individually charged with energy as desired by the clinician. Charging selected wires may be of particular advantage if the catheter sheath 12 comprises different sets of electrodes, such as electrodes 20 for ablation and electrodes 20 for sensing, mapping or diagnostics. It may also be advantageous to form an ablation zone or pattern based on activation of desired electrodes 20. Further, allowing charging or energisation of selected electrodes 20 will allow for improved procedures as a clinician may activate only selected electrodes 20.

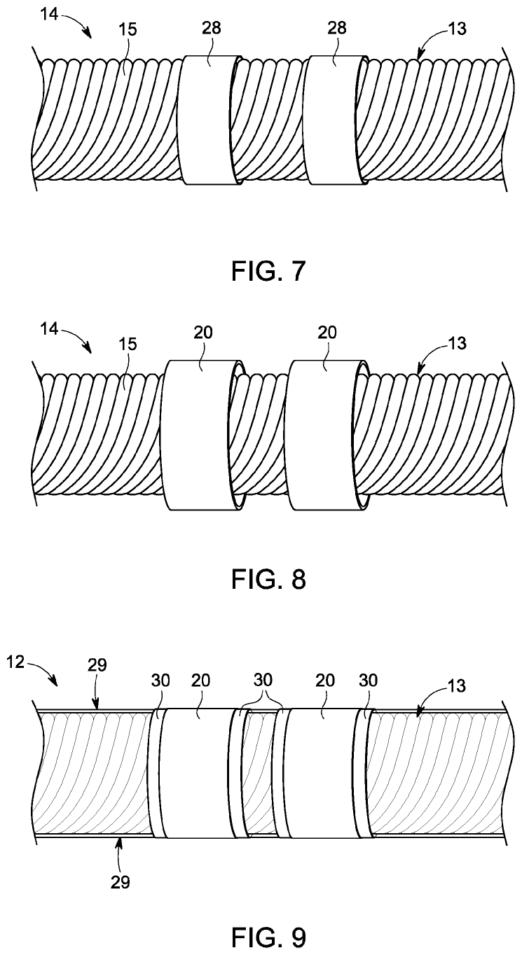

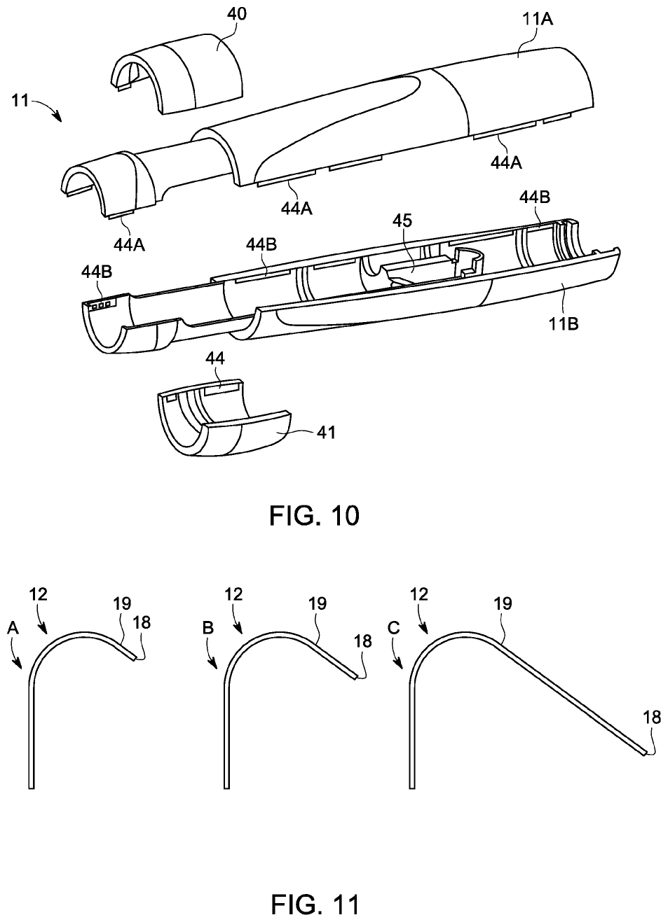

FIG. 6 depicts the apertures 22A filled with a conductive material 26 which contacts the conductive wire and extends the depth of the aperture 22A to the outer surface of the tubular member 14. The conductive material 26 may be a conductive fluid, such as a conductive adhesive which may further facilitate adhesion or fixing of a further component, such as a conductive band 28 or conductive ring. If the conductive material 26 is a deposited as a fluid, the fluid preferably hardens or solidifies after a time period. In another embodiment, electromagnetic radiation may be used to harden or solidify the conductive fluid. Optionally, if the catheter sheath 12 is to be used for transmission of fluids, further apertures 22 may be formed in the tubular member 14, in which the fluid apertures are not filled with conductive material 26, and are preferably formed after the application of the heat-shrink means (not shown). The fluid apertures (not shown) allow for fluid communication between a fluid lumen of the catheter sheath 12 and a target site. The target site may be organ tissue to be ablated or a location in vivo to be sensed via a diagnostic catheter sheath.

FIG. 7 shown an optional step in the method of manufacture, in which after the aperture 22 is filled, or at least partially filled, a conductive band 28 may be attached to the tubular member 14 at the location of a filled aperture 26. Each of the filled apertures may have a respective band 28 attached thereto. The conductive 28 band may be fixed to the tubular member 14 by the conductive material 26 if the conductive material is a conductive adhesive. The conductive bands 28 may be formed form a polymer, a metal, metal alloy or composite material. Alternatively, the conductive bands 28 may be printed onto the tubular member 14 or crimped to the tubular member 14. The conductive band may be adapted to allow for conduction of energy from the conductive material. It will be appreciated that the conductive band 28 is optional.

The electrode 20 is preferably a ring electrode 20 which can be axially mounted onto the tubular member 14. If the ring electrode 20 is adapted to be axially mounted onto the tubular member the ring electrode 20 preferably has an inner diameter which is larger than, or equal to, the outer diameter of the tubular member 14. A tight fit or snug fit between the electrode and the wound wires is preferred such that fluid cannot ingress between the electrode 20 and wires 15 in use.

Alternatively, the electrode 20 may instead be a split ring or a strip of conductive polymer which can be positioned around the tubular member 14 such that the strip or split ring end; meet, partially overlap or partially circumnavigate the tubular member 14 such that the strip or ring can be fixed to the tubular member 14 at a desired location. The split ring or strip may be fixed to the tubular member 14 by heat, adhesive, any other suitable fixing means or combination thereof. For example, the split ring or the strip may firstly secured in place with adhesive and then subsequently a heat treatment to fix the split ring or fix the strip in place.

Turning to FIG. 8, a heat-shrink means (not shown) is then applied over the tubular member 14 and the electrode 20. The heat-shrink means is preferably a tubular heat-shrink means which can be heat treated to shrink in a radially inward direction around the tubular member 14 and the electrode 20. The radial shrinking of the heat-shrink means is to fix the electrode 20 at a desired location on the tubular member 14. In this way the heat-shrink layer of the heat-chink means can act as a mould (mold). Preferably, the heat-shrink means comprises a heat shrink layer (not shown) and an intermediate layer 29 which comes into contact with the tubular member 14 and the electrodes 20 such that the intermediate layer 29 can be melted or become fluid during the time the heat-shrink layer shrinks. The order in which the shrinking and flowing is of importance as this fixes the electrodes 20 in a desired location, then allows for a flow of material to fill in gaps formed during application of the heat-shrink layer and form a generally uniform outer diameter. Flowing of the heat-shrink layer provides the advantage that the sides of the electrodes 20 come into full contact with the flowed material. This may form a linear outer surface of the catheter sheath 12 which is of particular advantage as there is a reduced risk with regards to lesions being formed inadvertently or undesirable cutting of anatomy of the patient.

It will be appreciated that an end cap or end electrode (not shown) may be fixed to the tubular member 14 prior to the application of the heat-shrink means. The end electrode or cap may be connected to a conductor 30 and may be adapted to be energised. The end cap may comprise a projection adapted to be inserted at least partially into a lumen 17 of the catheter lead 12, if at least one lumen 17 is present. Alternatively, the end cap may be attached after the application of the heat-shrink means and may be ultrasonically welded or induction heated/welded onto the catheter lead 12. It is preferred that the exterior of the catheter sheath is smooth or linear to reduce the potential for biological material to be deposited onto the catheter sheath 12 in use.

The intermediate layer 29 is preferably formed form a material which has a lower melting point than the shrinking point of the heat shrink layer, such that intermediate layer 29 material melts and flows as the heat-shrink deforms. Preferably, the intermediate layer 29 is a thermoset material such that after an initial heating the intermediate layer 29 does not deform, flow or melt with introduction of further heat. This may be essential as the intermediate layer 29 will form the exterior layer of the catheter sheath 12 which may be exposed to heat from ablation procedures.

In yet another embodiment, as the shrinking will radially deform generally uniformly when a uniform heat is applied, the outer diameter of the catheter lead 12 will be typically uniform after heat treatment. Heat may be applied via induction heating to allow for a flow of material and allow for improved control when applying heat to the catheter lead 12. Any mention of heating or welding in the present disclosure may include induction heating. As the electrodes 20 applied to the tubular member 14 are relatively elevated from the tubular member 14, a uniform or substantially uniform shrinking of the heat-shrink layer will cause the intermediate layer 29 to be forced into the regions between the electrodes 20, and therefore the outer surface of the electrodes will be generally free of the non-conductive material of the intermediate layer 29, and therefore allowing the electrodes 20 to function. The flow of the intermediate layer 29 will allow the material to flow and abut the side of the electrodes 20 at region 32. The region 32 on typical catheters will generally have a depression or other deformation which prevents a uniform outer diameter or prevents a flush/linear surface between the ring and the sheath (outer layer of material adjacent to the electrode) along the catheter lead 12. This typically occurs as the electrodes are clamped onto or mechanically urged onto the tubular member 14 or sheath 12. This is a significant issue with known catheters as this allows for biological material to be deposited in the deformation or depression, and further can cause snagging or catching of the catheter lead 12 during use. Therefore, it is an advantage to have a catheter lead 12 which has a uniform outer diameter, linear surface, or generally smooth outer surface. In addition, the flow of material to abut the sides of the electrode 20 at the regions 32 further assist with retaining the electrodes 20 at the correct location as axial movement is further prohibited or restricted.

In yet another embodiment, a bond is formed between the sides of the electrode 20 and the intermediate layer 29. This is advantageous as when the catheter 10 is shaped (i.e. flexed or bent), for example by a manipulation means during use, the bond at the region 32 prevents a gap or fissure from forming between the electrode 20 and the intermediate layer 29. Preferably, the modulus of elasticity (E) of the conductor and/or the intermediate layer 29 is sufficient to allow for elastic deformation during use.

It will be appreciated that the electrodes 20 may be formed with a patterned surface or undulating surface such that the intermediate material 29 may set in the troughs or pits of the pattern or undulation and leaving only the peaks or heightened areas of the electrode 20 exposed. This may allow for a desired electrode 20 shape or burn pattern to be formed when if the electrode is an ablation electrode 20.

In an unillustrated embodiment, the outer surface of the tubular member 14 may be formed with an electrode layer (not shown). The electrode layer preferably comprises an outer surface with undulations, or an array, or a pattern, or a texture with discernible peaks and troughs. The heat-shrink means may then be placed on the electrode layer to be heated, such that when heated, the intermediate layer 29 of the heat-shrink layer is at least in part displaced (flowed) based on the texture or undulations on the electrode layer. The displacement of the intermediate layer 29 urges the intermediate material into the troughs of the electrode layer, such that the peaks are exposed after removal of the heat-shrink layer.

After the heat-shrink means has been heated and the intermediate layer 29 has flowed to form a layer relatively above the wound wires 15 and adjacent the electrodes 20, the heat-shrink layer is removed. It will be appreciated that the intermediate layer 29 is a distinct portion with respect to the heat-shrink layer, such that the intermediate layer 29 can be deposited on the tubular member 14, while the heat-shrink layer can be removed without damaging the deposited intermediate layer 29. It will be appreciated that fluid apertures (not shown) may instead be formed at this time such that the fluid apertures are not filled with the intermediate material which has flowed during the heat treatment process.

In a further embodiment, after the electrode 20 has been positioned at a desired location on the tubular member 14 the electrode 20 is heat treated rather than applying a heat shrink layer with intermediate layer 29. This allows the electrode 20 to be fixed in a desired location without the use of a heat-shrink layer. The heat treatment of the electrode causes the ring electrode 20 to be fixed in place. Optionally, the electrode 20 is heat-shrinkable such that under application of a predetermined heat to the electrode 20 the electrode 20 shrinks to form a seal with the tubular member 14. When heated electrode 20 inner diameter shrinks to approximately the same diameter as that of the outer diameter of tubular member 14. After an initial heat treatment the electrode 20 is thermoset such that the ring does not deform or shrink under the application of further heat being applied, which allows for the electrode 20 to act as an ablation electrode if desired.

In yet a further embodiment, the tubular member 14 is manufactures by extrusion process. The tubular member 14 may comprise an outer jacket and an inner jacket (not shown) with at least one conductive element 30, such as a conductive wire 30, disposed between the outer jacket and the inner jacket. This process may allow for a more simplified construction of the tubular member 14 which may result in expedited manufacturing times. The conductive element preferably extends along the length of the tubular member towards the proximal end of the tubular member such that it can be connected to a power source. However, it will also be appreciated that the conductive element 30 may not extend to the proximal end of the tubular member 14, but instead be attached to a wire 15 or other connection means in which the connection means extends to the proximal end of the catheter lead 12. Optionally, an electrode 20, preferably a polymer electrode 20, may be welded, melted or fused onto the outer jacket. It will be appreciated that if the electrode 20 is welded, melted or fused onto the outer jacket the electrode 20 is adapted to receive energy or otherwise be in communication with at least one conductive element.

In yet another embodiment, the catheter lead 12 may be manufactured with at least one metal ring (not shown) around and/or adjacent to at least one electrode 20. The metal rings may be printed onto at least one electrode 20 to be used as an x-ray visible indicator, similar to that of a radiopaque marker. Optionally, a plurality of metal rings are disposed on desired electrodes such that the rings form a pattern or other marker which can be easily viewed from an x-ray to determine the orientation of portions of the lead 12. The metal rings may be swaged, induction heated, fused, adhered, fixed, crimpled or clamped onto the electrode 20. The metal rings may be applied after or before applying the heat-shrink means. The metal rings may be of varying thicknesses, shapes or configurations to allow for distinction between metal rings on the catheter lead 12.

After manufacture, the catheter sheath 12 may be attached to a catheter handle 11. A stylet may (not shown) also be inserted into the catheter sheath 12, preferably in the lumen 17 of the tubular member 14 such that the stylet can be used to impart a desired shape to the catheter sheath 12.

The catheter 10 may be a modular catheter 10 which comprises a number of modules within the catheter handle 11. The modules may be isolated to allow for selective removal or insertion into the catheter handle 11 to further enhance the utility of the catheter 10. The shell of the catheter handle 11 may be separable such that the components of the catheter handle 11 can be accessed. The prior art documents mentioned in this disclosure do not teach or disclose modules, but rather complex components contained in a catheter handle which are never intended to be removed and/or reprocessed.

The catheter handle 11 may comprise at least two shell portions 11A, 11B, illustrated as shell halves 11A. 11B which can be repeatedly separated and reattached. In one embodiment, the shell halves 11A. 11B comprise a hinge or bias to facilitate a predetermined mating of the shell halves 11A. 11B. The handle 11 may comprise a releasable mating means 44, such as a flanged projection 44A which mates with a corresponding flanged lip 44B. The flanged lips may be similar to that of a tabled splice joint. The catheter handle shell 11 is designed to allow for multiple connections and separations (or openings) of the catheter handle halves 11A. 11B. Preferably, if the catheter handle 11 is adapted to be opened the handle 11 may be adapted to only be opened by a specialised predetermined tool or key. The handle 11 may also be designed to break or deform if an incorrect tool is used to attempt to open the handle to serve as an anti-tampering mechanism. The catheter 10 may be optionally configured to deform or show evidence of tampering or prior use. Further, if the catheter handle 11 is adapted to be opened or separated, at least one of the halves or portions comprises a seal to reduce the potential for undesired fluids from entering into the handle or being expelled from the handle. For example, a gasket or seal (not shown) is provided about the periphery of at least one shell half 11A or 11B. Alternatively, the longitudinal axis of the handle may be fitted with a sealing means which prohibits ingress of fluids.

The handle 11 may be opened to allow for sterilisation of the interior of the handle, which is of particular advantage as catheters are generally not separable, not reprocessable, or comprise a number of obstructions within the handle which will increase difficulty of reprocessing. The device may further comprise a locking or one-way indicator to be actuated prior to use of the device. The one-way indicator may be used to visually show a clinician whether a device has previously been used. The indicator may be locked or fixed in place as the catheter is connected or before the catheter is connected to an energy source. In this way multiple uses of a catheter 10 may be avoided without proper sterilisation or reprocessing of a handle 11. It will be appreciated that separating the handle 11 may allow for the indicator to be reset such that the catheter 10 indicates that it is safe to use. Optionally, if the catheter handle is designed to be reprocessed, the handle may be marked (for example with a symbol or alphanumeric character) with a number or change colour based on the number of reprocessing procedures undertaken.

Optionally, the catheter sheath 12 needs to be separated from the catheter handle 11 prior to allowing opening or separation of the catheter handle 11. This prevents removal of modules from the catheter handle 11 or opening of the catheter handle 11 during use. Each module may have a predetermined mounting position in the catheter handle 11 with the mounting positions preferably also comprising a retaining means which is adapted to retain the modules in the catheter handle 11. A stylet may be insertable into the lumen of the catheter sheath 12 and may also be adapted to be withdrawn from the catheter handle 11.

The catheter sheath 12 comprises at least one electrode 20 on its outer surface. The electrode 20 may be used to energise fluid, sense for electrical impulses, or be used for heating tissue of an organ. The electrodes 20 may be ring electrodes, or more preferably the polymeric ring electrodes. Polymeric ring electrodes may be deposited onto the sheath 12 and be used instead of standard metal rings. Polymeric ring electrodes 20 may have the advantage that they can be deformed, at least in part, such that the catheter sheath 12 can be formed into any desired shape. Preferably, the polymeric rings 20 may be elastically deformed such that they return to a desired shape after deformation.

At least one electrode 22, such as a ring electrode or the like, may be disposed near to the distal end 18 of the catheter lead 12. Preferably, the catheter lead 12 comprises an array of electrodes 20 or a plurality of spaced apart electrodes 20 adapted to sense a target tissue location or electrical signals. In yet another embodiment, an electrode 20 may be adapted to detect or monitor temperature. The catheter lead 12 may be formed with a predetermined shape, such as a linear, non-rectilinear or loop shape. Optionally, an operator or clinician may alter the shape of the catheter lead 12. For example, the catheter lead 12 may be straightened, deflected or otherwise alter shape such that it may be adapted to trek through tortuous anatomy or be adapted for a superior abutting relationship with tissue of a patient.

The catheter sheath 12 of the present disclosure is preferably formed with plastic electrodes which allow for the electrode locations to bend, flex or otherwise change shape. Commonly, most catheter leads utilise a ring of platinum, platinum-iridium alloy, gold, or other biocompatible metal for the electrode 20, however these do not allow for the area in which the electrode 20 is attached to bend or flex, which may prevent the clinician forming the lead into a desired shape for tortuous anatomy. A polymer electrode 20 may be formed at least in part from at least one electron-conducting polymer selected from the group of; polyfluorene, polyphenylene, polypyrene, polyazulene, polynaphthalene, polypyrrole (PPY), polycarbazole, polyindole, polyazepine, polyaniline (PANI), polythiophene (PT), poly(3,4-ethylenedioxythiophene) (PEDOT), poly(p-phenylene sulfide) (PPS), Polyacetylene (PAC), Poly(p-phenylene vinylene) (PPV), or any other suitable conductive polymer. A polymeric material may allow for flexure, deformation or bending which cannot be achieved by conventional electrodes 20. Further there are difficulties with attachment of conventional electrodes, let alone conductive polymeric electrodes as described herein.

It will be appreciated that conductive polymers generally have poor processibility for commercial applications, however utilising the material in small portions as the present disclosure provides for may allow for these materials to be used effectively. A significant advantage of the present disclosure is the attachment method for the polymer conductor 20 with the use of the intermediate layer. Further, another suitable attachment method may be swaging or induction heating. It will be appreciated that the attachment methods discussed may also be suitable for metal components.

The intermediate layer may be formed from at least one polymer selected from the group of; Low-density polyethylene (LDPE), High-density polyethylene (HDPE), Polypropylene (PP), Polystyrene (PS), Polytetrafluoroethylene (PTFE), Polyvinylchloride (PVC), Polychlorotrifluoroethylene (PCTFE), enamel, polyurethane, polyester, nylon, polyimide or any other suitable polymer, such as any suitable biocompatible polymer. Other polymeric materials such as a polyether block amide (PEBAX) or silicone may be used.

The catheter lead 12 may be formed with any number of lumens 17. The exterior of the catheter sheath may further have components welded or fixed thereto, for example a fluid lumen may be ultrasonically welded to the catheter lead 12, or the intermediate layer maybe used to fuse, melt or attach the fluid lumen to the longitudinal axis of the catheter lead 12.

There are a number of different devices or components which allow for a desired shape to be imparted to the distal end of a catheter lead 12. A commonly used shape imparting means is a pull wire or a wire, which is fixed near to a distal end of the catheter lead 12 and may require an axial force applied to the pull wire to cause a shape to be imparted to the catheter lead 12 proximal the fixing location near to the distal end. There may be a number of problems associated with these shape imparting elements, such as they require at least a distal part of the pull wire to be fixed to the distal end, and therefore cannot allow for removal of the catheter lead 12 without first opening the catheter handle, in which the handle is typically welded or fixed in a closed position making this impossible at least without damaging the catheter handle 11. In addition, the pull wire can only impart a shape emanating from the fixing location of the wire on the catheter lead 12.

The catheter lead 12 may allow for a stylet to be inserted into a lumen 17 to impart a desired shape to the catheter lead 12. Unlike a pull-wire a stylet is not fixed to the catheter lead 12 and does not require tension to be applied to impart a shape to the catheter lead 12. This is a significant advantage as a shape can be imparted at and desired position of the catheter lead 12.

Helically winding 13 the conductors 15 may reduce kinks forming in the catheter lead 12 in use and may reduce the strain being imparted on the catheter lead 12 and/or handle. As such, a helical structure 13 may provide a more durable catheter 10 while also maintaining sufficient flexibility. It will be appreciated that multiple layers of helical winding 13 may be used for the manufacture a catheter lead 12. For example, the lead 12 may be formed with a first helical winding 13 with a second helical winding (not shown) relatively above and/or around the first helical winding 13. Preferably the first helical winding 13 has an outer diameter and the second helical winding has an inner diameter which is approximately the same as or greater than the outer diameter of the first helical winding 13. However, optionally the inner diameter of the second helical winding may be smaller than the outer diameter of the first helical winding 13 such that the second helical winding presses into the first helical winding 13, which may cause deformation of at least one of the first winding or the second winding and increase frictional forces between said first and second helical windings. This allows the second helical winding to be disposed around the first helical winding 13. Optionally, an intermediary layer can be provided between at least one of the windings. The intermediary layer (not shown) can be melted or flowed to allow for the windings to be embedded partially therein or allow for voids to be removed between respective windings and the intermediary layer. More than two helical windings and/or intermediary layers may be used to form the catheter lead 12. Having helical windings with opposing helix structures relative to an adjacent helical winding may allow for desirable torsion properties to be imparted to the catheter lead which can improve manipulation of the lead 12 in use. If multiple windings are used, the conductive wires may only be disposed in one winding (preferably the outer helical winding), however this is optional as apertures may be formed such that they extend through multiple windings and/or intermediary layers to arrive at a desired conductive element. Optionally, sensors or electrodes 20, such as diagnostics sensors or electrodes 20, may also be disposed under a winding or intermediate layer for use in diagnostics as this may reduce interference received by the diagnostics sensor/electrode 20 in use. Optionally, a lumen can be formed by at least one of the windings. It will also be appreciated that while the above embodiment refers to helical windings, at least one helical winding may instead be an extruded element with an outer jacket and an inner jacket with at least one conductor therebetween.

Referring to FIG. 11 there is shown an embodiment of a catheter lead 12 with a manipulation means (this may also be referred to as a shape imparting means throughout this specification) disposed inside the catheter lead 12 (which cannot be seen in FIG. 11), although reference number 19 represents the relative location of the distal end of the manipulation means 19 in the lead 12. The manipulation means may be at least one of a stylet, a guide wire, a shape imparting wire, an introducer an elongate rigid element or shape memory polymer any other suitable means for imparting a shape to a catheter lead 12. In this configuration the catheter lead 12 can be projected beyond the distal end of the stylet 19 generally with axial movement or displacement of the catheter lead 12 relative to the distal end 19 of the stylet. Axial displacement of the catheter lead 12 allows the shaped portion of the stylet to impart and/or maintain a desired shape in the catheter lead 12 which not being fixed to the catheter and further not imparting tension on the lead 12. It will be understood by a person of skill in the art that the function of a stylet is fundamentally different to that of a pull-wire. A pull-wire requires a tension to be applied which can move or undesirably shape a catheter lead, whereas a stylet has a shape imparted thereto which is mimicked or imparted to the catheter lead 12 as the stylet is within the lumen 19 of the catheter lead 12. Therefore, the axial displacement of the catheter lead 12 relative to the distal end of the stylet may allow a clinician to project or extend the catheter lead 12 beyond the distal end of the stylet 19. In this way the distal end 18 portion of the catheter lead 12 extending, beyond the distal end of the stylet 19 does not comprise a shape imparting element (such as a stylet) and therefore is free to project or be manoeuvred through tortuous anatomy more easily, which can reduce the potential for damaging anatomy and/or causing lesions during use. It will be appreciated that the stylet may remain stationary relative to the handle while the catheter sheath 12 is projected or axially displaced relative to that of the distal end of the stylet 19. Alternatively, the stylet may adapted to be at least partially withdrawn, or fully withdrawn, from the catheter 10 such that the distal portion of the catheter lead is free of the stylet, at least in part. The distal end of the stylet 19 may also be adapted to be substantially in register with the distal end of the catheter lead 18.

The stylet may be adapted to be axially withdrawn from the catheter 10 or axially displaced relative to the catheter handle 11. The catheter lead 12 may allow for insertion of more than one shape imparting element in a single lumen or respective lumens 17 within the catheter lead 12. The catheter lead 12 may comprise a fluid lumen or irrigation lumen 17 to allow for the passage of fluid through the catheter lead 12 to preferably the distal end portion 18 of the catheter lead. Apertures for delivery of fluid which are formed in the catheter lead 12 may be of a size which only allows for expulsion of fluid from the catheter lead 12 and prevents or restricts ingress of undesired fluids, such as blood. In addition, the fluid apertures may be a valved aperture which only allows for transfer of fluid between the fluid lumen of the catheter lead and external to the catheter under a predetermined pressure.

The present disclosure may provide for a manufacture of a catheter lead 12 which may eliminate or reduce the step height between the outer diameter of the catheter lead 12 and the electrode 20 thereon. This provides a number of significant mechanical and operational advantages and also reduces the potential for coagulation zones on the lead between the electrode and the catheter lead 12. This further reduces the potential for electrodes to be dislodged or come off during a procedure, which greatly improves safety.

Further, as a physical bond between a conductor and the electrode 20 may be formed, for example by conductive adhesive 26 or a conductive band 28, the connection to the electrodes is improved relative to conventional methods. Conventional methods generally require clamping or other compressional forces to attach an electrode 20 which can easily be disconnected in use causing ‘dropout’ of the electrodes making them useless or unfit for use in a procedure. Further, as catheter leads 12 are generally not replaceable in conventional catheters, this may lead to an entire catheter being discarded for a new catheter which is costly to the patient and/or hospital, and may also lead to complications during surgery and wasting surgeon or clinician time.

In addition, with the use of the heat-shrink means with an intermediate layer, the requirement for adhesives can be eliminated and therefore expediting production of a catheter lead while also improving the connection between an electrode and the catheter lead 12. Further, the use of polymer electrodes or electrodes formed partly therewith, can reduce costs and also production times.

In yet a further embodiment, the tubular member 14 includes conductors that are connected to the electrodes 20 at the distal end 18 of the tubular member 14. The conductors are able to carry the signal sensed by the electrodes 20 through the tubular member 14 to the handle 11 where the conductors 30 further connect to electrical instruments, for example, to a monitor, a stimulator, or a source of energy such as an RF energy source used for ablation. At least one conductor 30 may be associated with an electrode 20, but several conductors 30 may be required for one electrode 20. The tubular member 14 is preferably formed of a biocompatible and resilient material that is non-conductive. Polymeric materials such as a polyether block amide (PEBAX), silicone or polyurethane may be used.

The tubular member 14 is initially formed by an inner tubular section defining an elongate lumen 17 within the tubular member 14. A plurality of wires 15 are helically wound about an outer surface of the tubular member 14. If the wires do not comprise insulation or an outer jacket a coating (not shown) of an insulative material can be laid over the conductors 30 to sandwich, embed or compress the conductors 30 between the inner side of the coating.

One or more electrodes 20 may be attached to the distal end 18 of the tubular member 14 by forming an opening or aperture 22 in the insulative material forming outer wall of the tubular member 14. The aperture 22 is formed, for example, by laser cutting a portion out of the tubular member 14. Laser cutting facilitates accurate cutting of the tubular member 14 and only the desired conductor 30 or Conductors 30 can be revealed by cutting the portion out of the tubular member 14. The apertures 22 reveal one or more conductors 30 that are to be connected with an electrode 20. The conductor apertures 22A are filled with a suitable biocompatible adhesive 26 which is preferably also conductive. A electrode 20 may then slipped onto the tubular member to cover the apertures 22 so that the electrode 20 is adjacent to and surrounding the apertures 22. The adhesive 26 inserted into the opening is electrically conductive and, therefore, it ensures the conductive connection between the conductor 30 and the electrode 20.

In yet another embodiment, another type of tubular member 14 may be provided in which the tubular member 14 defines one or more elongate passages, lumens 17 which extend longitudinally through the tubular member 14. The lumens can be used, for example, to house a deflection stylet for a deflection type catheter, or one or more electrical conductors 30 extending through the tubular member 14 from the distal end 18 of the tubular member to the proximal end 16 of the tubular member. In this type of catheter sheath 14, the conductors run longitudinally within one of the lumens 17. To attach one or more electrodes 20 to the distal end 18 of the tubular member 14, an aperture 22 is formed on the outer wall of the tubular member 14 so that the conductor or conductors 30 may be drawn out of the tubular member to be connected to a respective electrode 20. The aperture 22 extends from the lumen 17 inside the tubular member 14 to the outside surface of the tubular member. Optionally, the conductor or conductors 30 are preferably connected to the inner surface of the electrode 20. This conductive connection between the conductors 30 and the electrode 20 is provided by welding, soldering or any other suitable method. Induction welding is preferred as it provides a consistent result. Once the conductor 30 is conductively connected to the electrode 20, the electrode 20 is axially mounted over the distal end 18 of the tubular member 14 to a position directly over the opening. The electrode 20 may otherwise be wrapped, clipped, crimped or attached by any other fixing means to the catheter lead 12.

Once the electrode 20 is slipped onto the tubular member 14 adjacent to and surrounding the apertures 22, it may be secured in place. Most conventional heating methods include an external heat source such as a hot air gun, or an oven. A method based on induction bonding has a number of advantages over these methods and it is, therefore, used to heat and melt the tubular member 14 surrounding the electrode 20 to form a seal between the electrode 20 and the apertures 22. Induction heating can be used to heat, melt or solder an electrically conducting article such as the electrode 20. The induction heater used to treat the heat-shrink means to fix it to the tubular member 12 may consist typically of a power supply that provides a high frequency alternating current that is passed through a coil. The tubular member 14 with the electrode 20 attached onto it is inserted through the coil. Current is induced within the electrode placed in the coil, causing resistive heating of the metal electrode 20. As the temperature of the electrode 20 increases, it melts the intermediate layer 29 of the heat-shrink means locally around the electrode 20 and bonds the two materials together. Induction heating is fast, clean and simpler to do than traditional methods to manufacture an electrode 20 assembly. Depending on the size of the coil, induction heating allows targeted heating to relatively small areas and is particularly useful for heating or soldering elongated rod-like articles. During the heating process, mandrels may be inserted into the lumen of the catheter lead 12 to support the tubular member and inhibit the collapse or deformation of the tubular member 14.

As the electrode 20 is slid onto the tubular member 14, a small gap remains between the electrode 20 outer surface and the tubular member 14 outer surface, also known as a “step”. This step can be filled by locally heating and melting the intermediate layer 29 around the electrode 20 to form a seal between electrode 20 and the tubular member 14. The region 30 bonds or fixes with provides enhanced bond strength and helps to prevent biological material from being caught adjacent to the electrode 20.

The bond between the electrode 20 and the tubular member 14 can be improved by swaging or another suitable mechanical compression method such as crimping (not shown). Swaging is a process for shaping metallic articles such as rods, bars, or tubes. In particular, it can be used to reduce the diameter of such articles, or producing a taper in them. Once the electrode 20 has been slid into its place covering the apertures 22, its diameter is reduced by swaging thus making the electrode tightly secure in its position on the tubular member 14. During swaging, the tubular member 14 with the electrode 20 is placed inside a die that applies compressive force by hammering and rotating around the ring. Alternatively, a mandrel can be inserted inside the tubular member during compression. Once the swaging process is complete, the outer diameter of the electrode 20 is substantially the same as the outer diameter of the tubular member 14.

The connection between the tubular member 14 and the electrode 20 when the electrode 20 has been secured in its place by swaging. Swaging may reduce the diameter of the electrode 20 and the electrode 20 has been compressed into the tubular member 14. Although the electrode 20 has been compressed into the tubular member 14 so as to be flush with the tubular member 14, a transition region 32 remains adjacent to the electrode 20 where the plastic tubular member 14 has been compressed. The transition region 32 runs circumferentially around the tubular member 14 in close proximity to the electrode 20. The tubular member 14 and the electrode 20 after the tubular member 14 with the electrode 20 has been exposed to induction heating. The small transition region 32 has been filled as the plastic material of the tubular member 14 has melted and solidified upon cooling tightly around the electrode 20. This provides a tight seal between the electrode 20 and the apertures 22 filled with conductive material 26.

A mould (mold) or die can further be used when manufacturing the catheter sheath 12 to ensure that the molten plastic of the tubular member 14 fills any gaps adjacent to the electrode 20, although this is preferably achieved by applying a heat-shrink means over the tubular member 14 and the electrodes 20 as discussed above. During the manufacturing process, the mould (mold) or die covers the tubular member 14 and the electrode 20 so that upon melting and cooling, the surface of the electrode becomes flush with the surface of the tubular member 14. As can be seen from FIG. 6b, the transition region 32 formed during swaging is filled with the plastic of the tubular member 14 after the induction heating has been effected and the electrode 20 is flush with the outside surface of the tubular member 14. The term ‘flush’ may be interpreted as being level or even with another surface, such that there is a smooth surface between two at least two elements. For example, the outer surface of the electrode 20 may be level with the intermediate layer 29.

In an unillustrated embodiment, a heat-shrink means or other suitable tubing may be placed over the electrode 20. Upon induction heating, the electrode 20 conducts heat to the heat-shrink means, which in turn melts and moulds (molds) the plastic tubular member 14 so that the outer surface of the electrode 20 is flush with the surface of the tubular member 14. Heating of the tubular member 14 causes the material of the tubular member 14 to liquefy to an extent and to flow causing region 32 to form which is preferably substantially linear or free of steps or locations in which biological material can be trapped or deposited easily.

The advantage of the present catheter sheath 12 and its manufacturing method is that it provides a tight seal between the electrode 20 and the tubular member 14, resulting in no fluid or other substance to be able to pass underneath the electrode 20. It is a further advantage that adhesion (or bonding) between the electrode 20 and the tubular member 14 is enhanced without using adhesives. Treating the tubular member 14 and the electrode 20 by induction heating further assures a smooth transition between the outer circumferential surface of the electrode 20 and the outer circumferential surface of the tubular member 14. It is a further advantage that the method of manufacturing of the catheter sheath simplifies the procedure of producing a suitable catheter sheath 12. In addition, the use of the induction heating technique to cause flow of the material of the tubular sheath 14 assists in sealing the tubular member 14 against the ingress of foreign material. This heating technique also serves to assist in retaining the electrodes 20 at a desired location on the catheter lead 12.

Optionally, the heat-shrink layer of the heat-shrink means may comprise a conductive polymeric material. This may allow for induction heating to be used to shrink the heat-shrink layer. The heat applied to the heat-shrink layer can be transferred to the intermediate layer 29 to fluidise, cause flow of or otherwise melt the intermediate layer 29 to form the outer surface of the catheter lead 12.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

1. A catheter lead for a procedure, the catheter lead comprising;
a tubular member with at least one conductive wire;
the tubular member having a proximal end and a distal end;
the distal end of the catheter comprising at least one electrode in communication with the conductive wire; and
wherein the at least one electrode comprises a conductive polymeric material.

2. The catheter sheath as claimed in claim 1, wherein the at least one conductive wire is helically wound.

3. The catheter sheath as claimed in claim 2, the at least one conductive wire is helically wound around the tubular member.

4. The catheter sheath as claimed in claim 2, wherein the tubular member is formed from the helically wound at least one conductive wire.

5. The catheter sheath as claimed in any one of the preceding claims, wherein the electrode is adapted to deform.

6. The catheter sheath as claimed in any one of the preceding claims, wherein the electrode is adapted to elastically deform.

7. The catheter sheath as claimed in any one of the preceding claims, wherein the electrode is formed from conductive polymeric material.

8. The catheter sheath as claimed in any one of the preceding claims, wherein the distal end of the catheter comprises at least two electrodes in which an intermediate layer disposed between the at least two electrodes.

9. The catheter sheath as claimed in claim 8, wherein the intermediate layer abuts a side of the at least two electrodes.

10. The catheter sheath as claimed in claim 8 or claim 9, wherein the outer surface of the intermediate layer and the outer surface of the at least two electrodes is flush.

11. The catheter sheath as claimed in any one of the preceding claims, wherein the outer surface of the catheter lead is molded by a heat-shrink layer.

12. A method of manufacturing a catheter sheath, the method comprising the steps of;
cutting an aperture in a tubular member to expose a conductive wire;
applying an electrode over the conductive wire;
applying a heat-shrink layer with an intermediate layer over the tubular member and the electrode;
applying heat to the heat-shrink layer such that the intermediate layer transitions from a solid state to a fluid state, the heat further causing the heat-shrink layer to radially shrink around the intermediate layer; and
removing the heat-shrink layer after the intermediate layer has solidified.

13. The catheter sheath as claimed in any one of the preceding claims, wherein the aperture the expose the conductive wire is filled with a conductive material.

14. The catheter sheath as claimed in any one of the preceding claims, wherein at least one tacking aperture is formed in the tubular member.

15. The catheter sheath as claimed in any one of the preceding claims, wherein a conductive band is disposed between the electrode and the conductive wire.

16. The catheter sheath as claimed in any one of the preceding claims, wherein the outer surface of the intermediate layer is moulded to be substantially level with the outer surface the electrode.

17. The catheter sheath as claimed in claim 16, wherein the heat-shrink layer is used as a mould to mould the intermediate layer.
